(19) United States
(12) Patent Application Publication
(10) Pub. No.: US 2022/0246252 A1
(43) Pub. Date: Aug. 4, 2022

(54) METHOD FOR PRODUCING THERMOPLASTIC COMPOSITIONS FOR MECHANICALLY AND/OR THERMALLY STRESSED COMPONENTS

(71) Applicant: Evonik Operations GmbH, Essen (DE)

(72) Inventors: Wiebke Stache, Herten (DE); Frank Weinelt, Billerbeck (DE)

(73) Assignee: Evonik Operations GmbH, Essen (DE)

(21) Appl. No.: 17/642,562

(22) PCT Filed: Sep. 4, 2020
(86) PCT No.: PCT/EP2020/074747

§ 371 Date: Mar. 11, 2022

(30) Foreign Application Priority Data
Oct. 1, 2019 (EP) 19200877.9

(51) Int. Cl. G16C 60/00; G16C 20/30; G16C 20/70

(52) U.S. Cl. CPC G16C 60/00; G16C 20/70; G16C 20/30

(57) ABSTRACT

The invention relates to a method for generating thermoplastic compositions for mechanically and/or thermally loaded component parts.

### CROSS REFERENCE TO RELATED APPLICATIONS

This application is a 35 U.S.C. § 371 U.S. national phase entry of International Application No. PCT/EP2020/074747 having an international filing date of Sep. 4, 2020, which claims the benefit of European Application No. 19200877.9 filed Oct. 1, 2019, each of which is incorporated herein by reference in its entirety.

### FIELD

The invention relates to a method for generating thermoplastic compositions for mechanically and/or thermally loaded component parts.

### BACKGROUND

Compositions for mechanically and/or thermally loaded component parts are complex mixtures of raw materials. Customary compositions or formulas or compounds for mechanically and/or thermally loaded component parts contain around 20 raw materials, also called “components” below. These compositions consist, for example, of raw materials selected from polymers, processing aids, stabilizers, colorants, conductivity aids (electrical), thermal conductivity additives, reinforcing agents, impact resistance modifiers; fillers; flame retardants and plasticizers.

New compositions and compounds having particular, desired properties have hitherto been specified on the basis of empirical values, and produced and tested accordingly. The constitution of a new composition which meets particular expectations of its chemical, physical, optical, tactile and other properties which can be captured metrologically is almost impossible to forecast, even for a skilled person, owing to the complexity of the interactions. The diversity of the interactions of the raw materials with one another and, in consequence, the large number of failed attempts involved, make this approach both time-consuming and expensive. The properties for the same make-up of the composition are also optionally dependent on the production conditions.

US 2018/0276348 A1 discloses a cognitive computer system for producing chemical formulations. The system determines a chemical formulation meeting certain restrictions, and produces and tests the chemical formulation. This computer system is based on the training of a system which learns, using existing data for chemical formulations. Compiling sufficiently large data sets in order to train a learning logic system using this data, however, is very complicated and also expensive in light of the large amount of time and materials involved. In many cases, as well, it is not possible simply to employ a data set that exists in the majority of laboratories for compositions that have already been produced and analyzed. There may be various reasons for this: the laboratory has been newly set up, and as yet does not possess any such data pool. The laboratory is establishing a new product line and as yet has no experience or corresponding data sets relating to the properties of this new product line. Or else the data which does exist is too narrow in scope or too biased, in terms of its historical composition, to be able to be used as a training data set.

Consequently there are highly confined limits currently imposed on estimation and prediction, either carried out by a human or else computer assisted, in relation to the components of a composition having desired properties. This is especially true of complex compositions having numerous relevant properties and numerous components, as is the case with thermoplastic compositions for mechanically and/or thermally loaded component parts, since the components interact with one another in a complex way and determine the properties of the corresponding chemical products.

At present, therefore, new compositions must first be produced as real chemicals, and their properties then measured, in order to allow an estimation of whether the compositions exhibit particular required properties. While there are already approaches to the automatic forecasting of properties of chemical substances, the compilation of a training data set of sufficient size and quality is nevertheless still often more complicated than the direct production and testing of the composition in question. The development of new thermoplastic compositions for mechanically and/or thermally loaded component parts is particularly complicated and requires a great deal of time.

It is therefore the object of the present invention to provide a method by which a new composition is developed or a compound is developed in a simpler, more time-saving and cost-effective way.

### SUMMARY

The present invention is directed to a method for generating a thermoplastic composition for mechanically and/or thermally loaded component parts, wherein the components of the composition have one or more polymers such as polyamides, polyesters, polyolefins, polycarbonates and polyaryletherketones, where the composition is generated by a computer system (224), where the computer system has access to a database (204), where known compositions (206) are stored with their components and properties in the database, and where the computer system is connected to a facility (244) for producing and testing compositions for mechanically and/or thermally loaded component parts, where the computer system comprises a neural network (226) and an active learning module (222).

### BRIEF DESCRIPTION OF DRAWINGS

FIG. 1 shows a flow diagram of a method for training a neural network and for using the trained network to predict properties and/or to predict a composition of a liquid medium.

FIG. 2 shows a block diagram of a distributed system for training a neural network and for using the trained network.

FIG. 3 shows a 2D detail of a multi-dimensional data space from which the active learning module selects data points in a targeted way

FIG. 4 shows the architecture of a neural network with input and output vectors.

The object is achieved by the method for generating thermoplastic compositions for mechanically and/or thermally loaded component parts, and also by a corresponding computer system and computer program product. Embodiments of the invention are indicated in the dependent claims. Embodiments of the present invention may be freely combined with one another, provided that they are not mutually exclusive.

The loading of the claimed components furthermore also comprises loading by chemicals and weather.

In one aspect the invention relates to a method for generating thermoplastic compositions for mechanically and/or thermally loaded component parts. The composition is generated by a computer system. The computer system has access to a database in which known compositions are stored with their components and properties. The computer system is connected to a facility for producing and testing thermoplastic compositions. The computer system comprises a neural network and an active learning module. The method comprises the following steps:
a. use of known compositions stored in the database for training the neural network, where a loss function is minimized for the training,
b. testing to determine whether the value of the loss function meets a specified criterion, where selectively, in the event that the criterion is not met, the following steps are carried out:
   i. selection of a trial composition from a quantity of specified trial compositions by the active learning module,
   ii. initiating of the facility by the computer system for producing and testing the selected trial composition,
   iii. training of the neural network using the selected trial composition and the properties thereof captured by the facility,
   iv. repeated implementation of step b,
c. generation of a forecast composition for mechanically and/or thermally loaded component parts by input of an input vector into the neural network,
d. output of the forecast composition by the neural network.

The neural network can for example be a multi-layer perceptron (MLP), a convolutional neural network (CNN), a recurrent neural network (RNN), memory-augmented neural network (MANN) and tree-recursive neural network or else bring together combined methods (hybrid/complex models) such as, for example, generative adversarial networks (GAN), named entity recognition (NER) and/or deep reinforcement learning.

Preferably, the neural network contains a recurrent network, a convolutional neural network or the combination of the two; more preferably the neural network is a recurrent network, a convolutional neural network or the combination of the two.

This is advantageous since, in a fully automatic or semi-automatic iterative method, an existing training data set is expanded in steps and in a targeted way to include meaningful additional training data in the form of trial compositions and their empirically determined properties, the neural network being trained anew on each iteration with the expanded training data set, thereby improving the predictive quality of the network after each iteration, until the loss function fulfils the criterion—that is, until a forecasting error of the network has become sufficiently small.

Because of the limited number of compositions that are already known, the trained neural network obtained after the initial training phase is often not yet in a position to predict reliably a forecast composition which exhibits a series of desired properties. For this, the data pool used is often too small.

Extending the training data set by an active learning module makes it possible to select a few trial compositions in a targeted way, to the particular profit of the quality of the neural network and its predictions. In a plurality of iterations, accordingly, through automatic and through targeted selection of trial compositions which promise particularly great improvement of the predictive model of the neural network, and also automatically or manually executed synthesis and analytical steps based on the particular trial composition selected, it is possible to achieve rapid improvement in the predictive power of the neural network in an efficient way. If after an iteration it is found that the loss function fulfils the criterion, it is no longer necessary to expand the training data set, since the predictive power of the trained neural network can be considered to be sufficient.

In a further advantageous aspect, the active learning module is able, through automatic selection of trial compositions which particularly boost the predictive power of the neural network, to expand existing data stocks of known compositions in such a way that any lack of balance on the part of the training data can be compensated largely automatically.

According to embodiments of the invention, the quantity of the trial compositions is generated automatically by a trial planning program. For example, the trial compositions can be generated automatically on the basis of the known compositions by addition or omission of components or by the modification of the amount and/or concentration of one or more components.

This may be advantageous since a very large number of trial compositions can be generated automatically. It is possible accordingly to cover a large data space with candidate compositions. The data space covered by the trial compositions may in particular have the advantage that it is less “biased”, if, for example, the trial planning program is designed to generate the trial compositions in such a way that substantially each component is subjected to similar variations (removing, adding, changing concentration), which cover a broad data space, so as to generate the trial compositions.

Thermoplastics are plastics which can be (thermoplastically) deformed within a defined temperature range. This process is reversible, meaning that it can be repeated ad infinitum by cooling and reheating to the liquid melt state.

According to embodiments of the invention, the components of the known compositions and/or of the trial compositions are consisting of one or more polymers such as polyamide, polyesters, polyolefins, polycarbonates and polyaryletherketones; and optionally further components such as processing assistants such as lubricants, flow aids, nucleating agents and demoulding aids;

stabilizers such as heat stabilizers, antioxidants, light stabilizers and UV absorbers; colorants such as dyes, pigments and optical brighteners; conductivity assistants (electrical) such as conductive carbon blacks, carbon nanotubes, graphene, ionic liquids or complex salts; thermal conductivity additives such as alumina or boron nitride; reinforcing agents such as glass fibres and carbon fibers; impact modifiers; fillers; flame retardants; and plasticizers.

According to embodiments, production comprises optionally the chemical synthesis of the components such as that of the polymers, and also the production of the compositions from the components.

According to embodiments of the invention, the optional production possibilities are selected from the group consisting of extrusion and injection moulding, with the production parameters being selected from machine data, barrel temperatures, injection profile, switchover travel, switchover pressure (hydr.), internal mould pressure on switchover, hold pressure profile, hold pressure time, hold pressure (hydr.), plastication, metering stroke (vPLAST), metering speed, metering stroke (pSTAU), back pressure (hydr.), residual mass cushion, residual cooling time, max. internal mould pressure pN, metering time, metering delay time, release after metering, release speed, metering stroke (actual), cycle time, metering stroke, closing force, intake zone temperature, torque, screw configuration, pressure, torque maximum, melt filtration, devolatilization, after-drying temperature, batch quantity, barrel temperature, throughput, melt temperature, die temperature, vacuum devolatilization, after-drying time, after-drying atmosphere, barrel zone, rotary speed.

According to embodiments of the invention, the properties of the trial composition (compounds) that are detected by the facility are selected from the group consisting of notched impact toughness, bursting periphery tension, BET surface area, amino end-group content, carboxyl end-group content, density, dielectric constant, conventional bowing, failure mode, yield stress, breakdown resistance, maximum penetration force, number of fractures—ball drop and hammer drop tests, color number Lab, pull-out force (fittings), corrected intrinsic viscosity (CIV), permeation (time), melt-volume flow rate (MVR), oxygen index, surface resistance, melt viscosity, end-group total, melting temperature, enthalpy of fusion, glass transition temperature, crystallinity, dimensional change, tapped density, transmittance, combustibility (UL94), Vicat softening temperature, heat distortion resistance temperature and water content, breaking stress, elongation at break, elasticity modulus, heat resistance.

According to embodiments of the invention, the forecast composition is output on a user interface of the computer system. The user interface may be, for example, a display, a speaker and/or a printer.

This may be advantageous since the user is able to check the forecast composition once again manually for plausibility before it is transmitted to the chemical facility for the purpose of production.

According to embodiments, the facility comprises at least two workstations.

According to embodiments, the method further comprises: input of a composition to a processor which controls the facility, where the composition input into the processor is the selected trial composition or the forecast composition, where the processor drives the facility to produce the input composition, where in the at least two workstations the input composition is produced and the properties of the input composition are measured, after which the measured properties are output on a user interface of the computer system and/or the measured properties are stored in the database.

The iterative synthesis and testing (determination of the properties) of the trial compositions in order to expand the training data set may be advantageous since a fully automatic or—if user confirmation is required—semi-automatic system is provided for the targeted expansion of a defined, already existing training data set for the iterative improvement of a neural network. Accordingly, the prediction method based on the neural network improves itself iteratively by corresponding control of the chemical facility and automatic use of the empirical data thus generated for the purpose of expanding the training data set.

The synthesis and testing (determination of the properties) of the forecast composition may be advantageous since a system is provided in which a user need specify only the desired properties of the chemical product; the determination of the components required for this product, and the generation of the product having the desired properties, take place, provided that the neural network has been able to determine a forecast composition for the required properties specified in the input vector.

According to embodiments, the computer system is configured to communicate via communications interfaces with the database and/or the facility for producing and testing thermoplastic compositions for mechanically and/or thermally loaded component parts. Communications interfaces are known to those skilled in the art, for example SCSI, USB, FireWire, Bluetooth, Ethernet, WLAN, LAN or other network interfaces.

According to embodiments, the compositions comprise or consist of compounds.

In a further aspect, the invention relates to a computer system for generating thermoplastic compositions for mechanically and/or thermally loaded component parts. The computer system comprises a database and a user interface and is configured to implement a method for generating a composition according to embodiments of the invention.

In a further aspect, the invention relates to a computer program, a digital storage medium or computer program product with instructions which can be executed by a processor and which, when executed by the processor, cause the latter to implement a method for generating a composition according to embodiments of the invention.

In a further aspect the invention relates to a system which comprises the said computer system and a facility. The facility is a facility for producing and testing compositions for mechanically and/or thermally loaded component parts. The facility comprises at least two workstations.

A “composition” refers here to a chemical product specification which specifies at least the nature of the raw materials (“components”) from which the chemical product is formed. Any reference in the context of this application to the production or testing of a composition should be understood as a short-form way of saying that a chemical product is being produced in accordance with the details specified in the summary in relation to the components and also, optionally, the concentrations thereof, or, respectively, that this chemical product is being “tested”—that is, its properties are being captured metrologically.

A “compound” here refers to a composition which as well as the statement of the component identity and the quantity details or concentration details of the components also comprises the production conditions.

A “known composition” is a composition which specifies a chemical product whose properties, at the time of training of a neural network, are known to the organization or person conducting the training, since the known composition has already been used once to produce a chemical product, and the properties of that product have been measured empirically. The measurement need not necessarily have been carried out by the operator of the chemical laboratory which is now determining the forecast composition; instead, it may also have been carried out and published by other laboratories, and so in this case the properties are taken from the specialist literature. Since, according to the definition above, a composition also includes compounds as a sub-quantity, the “known compositions” according to embodiments of the invention may also comprise “known compounds” or be “known compounds”.

A “trial composition” refers to a composition which specifies a chemical product whose properties, at the time of the training of a neural network, are not known to the organization or person conducting the training. For example, a trial composition may be a composition which has been specified manually or automatically but which has not yet been used for actual production of a corresponding chemical product either. Accordingly, the properties of this product are also not known. Since, according to the definition above, a composition also includes compounds as a sub-quantity, the “trial compositions” according to embodiments of the invention may also comprise “trial compounds” or be “trial compounds”.

A “forecast composition” is understood here to be a composition in respect of which a trained neural network predicts (forecasts) that it specifies a chemical product whose properties correspond to a specification of desired properties as mandated by a user. The specification of the desired properties may be provided to the neural network, for example, as an input vector which indicates, for each of the desired properties, a desired or acceptable parameter value or parameter value range.

A “database” here refers to any data storage facility or memory region in which data are stored, especially structured data. The database may comprise one or more text files, spreadsheet files, a directory in a directory tree, or a database of a relational or object-oriented structured database management system (DBMS), e.g. SQL such as MySQL or PostgreSQL; XML or noSQL such as N1QL or JSON.

A “loss function” (also called “target function”) of a prediction problem is a function which is used in the training of a neural network and which outputs a value whose amount provides an indication of the quality of the predictive model of the trained neural network and which is to be minimized in the course of the training, because the amount of this value indicates the erroneousness of the predictions of the neural network.

A “facility” for producing and testing compositions refers here to a system which consists of a plurality of laboratory equipment items and optionally a transport unit and which is capable of jointly controlling the laboratory equipment items and the transport unit in an orchestrated way in order to carry out, automatically or semi-automatically, a chemical workflow. The workflow may be, for example, a production workflow or an analysis workflow or a combination of both workflows.

“Testing compositions” by means of the facility refers to the metrological capture (“analysis”) of properties of a chemical product that has been generated in accordance with the details in the composition.

An “active learning module” is a software program, or a module of a software program, which is designed to select, in a targeted way, a (comparatively small) sub-quantity of trial compositions from a quantity of trial compositions in such a way that, after synthesis and empirical measurement of the properties of this selected trial composition and after this data has been taken into account when training the neural network, a particularly strong learning effect occurs.

In the figures below, embodiments of the invention are elucidated exemplarily in more detail:

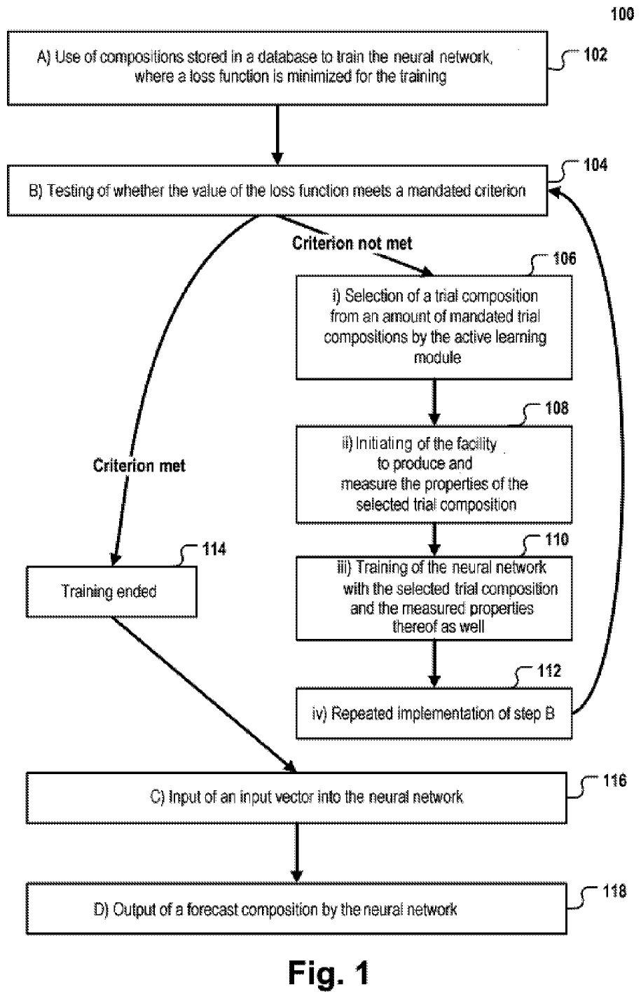

FIG. 1 shows a flow diagram of a method for training a neural network and for using the trained network to predict properties and/or to predict a composition of a liquid medium;

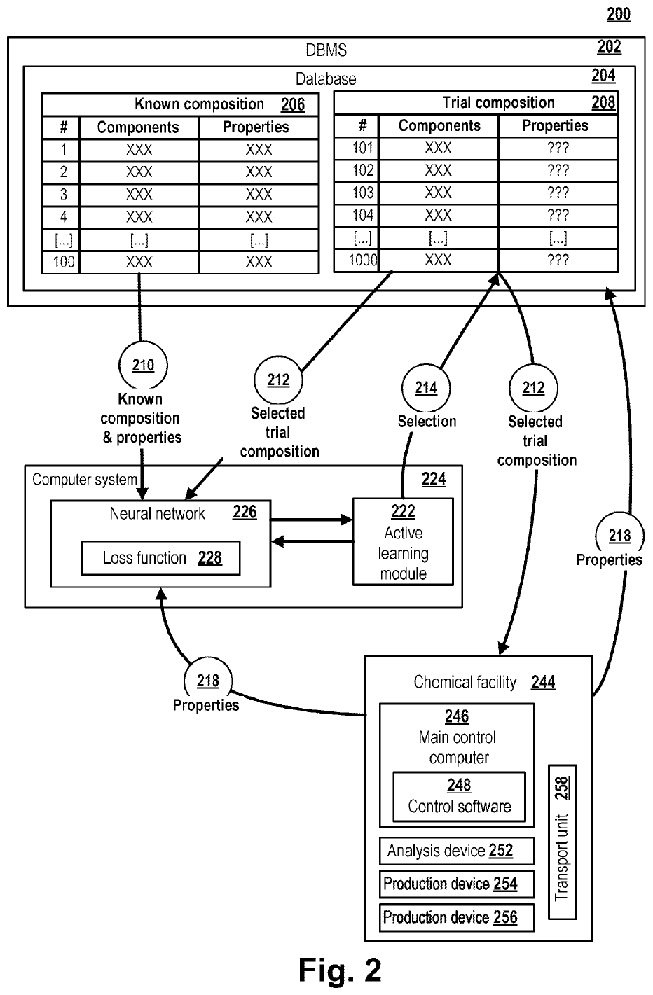

FIG. 2 shows a block diagram of a distributed system for training a neural network and for using the trained network;

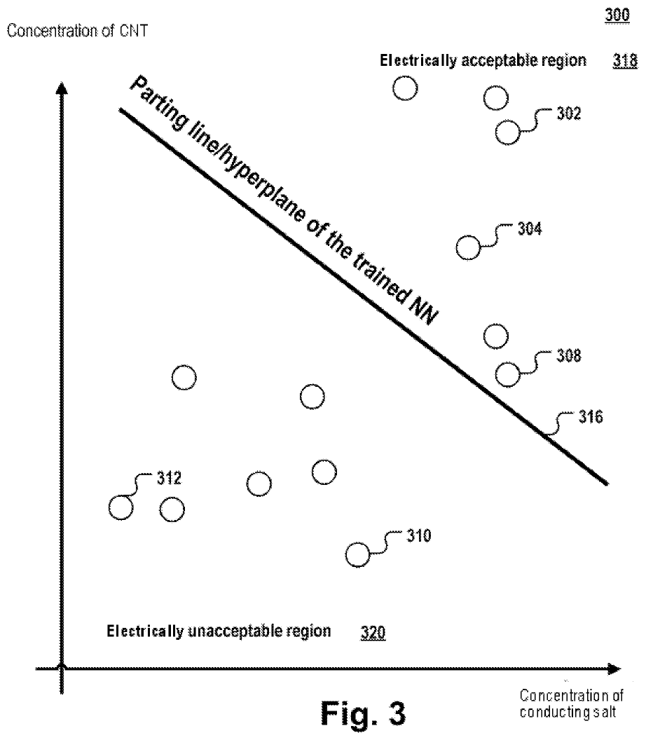

FIG. 3 shows a 2D detail of a multi-dimensional data space from which the active learning module selects data points in a targeted way;

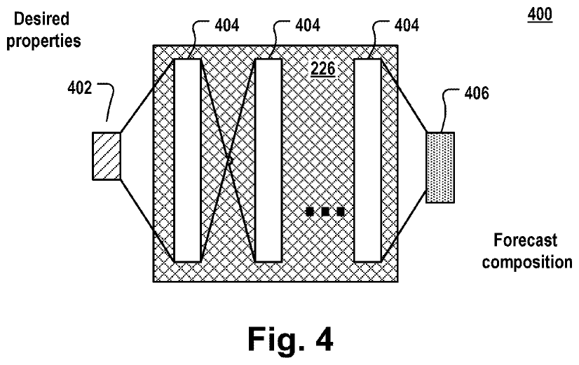

FIG. 4 shows the architecture of a neural network with input and output vectors.

FIG. 1 shows a flow diagram of a computer-implemented method for generating thermoplastic compositions for mechanically and/or thermally loaded component parts. The method may be executed, for example, by a computer system 224 as represented in FIG. 2.

In a first step 102 (a) already known compositions are used as an “initial training data set” in order to train a neural network in such a way that in response to the receipt of an input vector comprising one or more desired properties of a chemical product of the categories designated above, it predicts a forecast composition which has these desired properties. The forecast composition specifies at least the nature of the components of which a chemical product of the aforementioned kind (component parts) consists, and optionally the respective amounts and/or concentrations thereof as well. A combination of a known composition with the already known, empirically determined properties of the chemical product specified by this known composition represents an individual data point or data set within the entirety of the initial training data.

In the next step 104 (b) a check is made as to whether a value of a loss function fulfils a mandated criterion. Fulfilment of the criterion expresses the fact that the predictive accuracy of the trained neural network is to be regarded as sufficient. Selectively, in the event that the criterion is not fulfilled, the steps 106-112 outlined below are carried out. Otherwise, the training is ended (step 114) and the fully trained neural network is returned.

In step 106, the active learning module automatically selects a trial composition from a quantity of mandated trial compositions. There are a large number of different active learning approaches which can be used according to embodiments of the invention.

According to one implementation variant, the active learning module follows the “Expected model change” approach and selects the trial composition which (on retraining of the network taking account of this trial composition and its realistically measured properties) would bring about the greatest change in the current predictive model of the trained neural network.

According to another implementation variant, the active learning module follows the “Expected error reduction” approach and selects the trial composition which would most greatly reduce an error of the current predictive module of the trained neural network.

According to a further implementation variant, the active learning module follows the “Minimum Marginal Hyperplane” approach and selects the trial composition which lies closest to a parting line or parting plane which in a multi-dimensional data space is generated by the current predictive model of the trained neural network. The parting line or parting plane comprises interfaces within the multi-dimensional data space in which the predictive model makes a classifying decision, i.e. assigns data points on one side of the parting line or parting plane to a different class or category from the data points on the other side of the parting line. This proximity of the data points to the parting plane is interpreted to mean that the predictive model is unsure as to a classifying decision and would benefit particularly greatly from additional measurement of real-life data sets (consisting of a combination of components and optionally their concentrations and the measured properties of the chemical product generated according to this composition of the components) from the proximity of this parting plane, in order thereby to carry out further training of the neural network.

After the selection of one of the trial compositions from the database, the computer system, in step 108, initiates a facility for producing and testing chemical compositions, in such a way as to produce and test optionally automatically a chemical product according to the details in the selected trial composition. This testing is understood as meaning the metrological capture of one or more properties of the chemical product—in other words, for example, the measurement of amino end-group content, carboxyl end-group content, breakdown resistance, melt-volume flow rate (MVR), glass transition temperature, combustibility (UL94), notched impact toughness, breaking stress, elongation at break, yield stress, elasticity modulus, heat resistance or the like.

The properties measured in real life and obtained in step 108 are used in order to supplement the selected trial composition so as to form a complete further data point consisting of a known composition and known properties, which serves to expand the training data set used in a) and/or previous iterations. In step 110, the neural network is therefore retrained using an extended training data set. According to the implementation variant, this may be done such that the training is undertaken completely once again on the basis of the expanded training data set, or the training in step 110 takes place incrementally, so that what has been learnt before is retained and is only modified by the consideration of the new training data point.

In step 112, repeated testing of the predictive quality of the trained neural network is initiated, and steps 104-112 are repeated until the network possesses sufficient predictive quality, as evident from the fact that the loss function fulfils the criterion, in other words, for example, the “error value” calculated by the loss function is below a pre-defined maximum value.

The fully trained neural network can then be used for very rapidly and reliably predicting compositions which have one or more desired properties (a so-called “forecast composition”). For this purpose, a user in step 116 inputs an input vector into the trained neural network that specifies one or more of the desired properties. For example, the elements of the input vector may consist of a numerical value or value range which is to be understood as a desired or acceptable value or value range. A requirement, for example, may be to produce a composition having a conductivity in a defined value range and a color in a defined color range.

Since the network, in a plurality of iterations, based on a rationally and targetedly expanded training data set, has learnt the statistical correlations between components (and optionally their concentrations as well) and the properties of the resulting chemical product, the trained neural network is then able in step 118 to predict a forecast composition which has the desired properties, and to output this formulation to the user and/or to a chemical facility for direct synthesis.

The aim of training the neural network is so that the trained network, on the basis of an input amount of desired properties and corresponding parameter value ranges, is able to predict a forecast composition, in other words a specification at least of the nature and number and optionally the respective amounts as well of the components of a chemical product which has the desired properties. On applying the prediction to a test composition whose components are known and whose actual properties have been measured empirically, therefore, there is either a right or wrong forecast result. The forecasting quality of a trained neural network is assessed using the “loss function”, as mentioned above.

A “loss function” (also called “target function”) for a prediction problem, which may also be understood as a classification problem, may in the simplest case, for example, count only the correctly recognized predictions from an amount of predictions. The higher the proportion of the correct predictions, the higher the quality of the predictive model of the trained neural network.

For example, the question of whether an electrical property such as the conductivity lies within a pre-defined acceptable range may be understood as a classification problem.

There are, however, also numerous alternative possible loss functions and corresponding criteria for assessing the predictive accuracy of the trained neural network. For example, the network may receive desired properties in the form of an input vector and may use this data to predict the nature of the components of the composition. In these application scenarios, the neural network is supposed to estimate a value and not a class, in other words, for example, the amount of a component of a composition. These application scenarios are called regression problems. They require a different target function. For example, a loss function for regression problems may be a function which calculates the degree to which the value predicted by the network differs from the value actually measured. For example, the loss function may be a function whose output value correlates positively with the aggregated value of the deviation of every predicted component from a component actually used in a produced composition. The aggregated value may be an arithmetic mean, for example. For example, the quality of a trained neural network may be considered to be sufficient if these aggregated deviations (errors) of all components of a composition that are predicted by the network lie below a pre-defined threshold value with regard to the components actually used. In this case, the neural network may be used to predict unknown compositions which specify a chemical product having one or more desired properties. If these aggregated deviations of the predicted components from the components actually used deviate to a greater extent, on the other hand, than in the mandated maximum value, the active learning module is automatically caused to expand the training data set by selecting a further trial composition, causing the chemical facility to synthesize this composition and analyze its properties, and using the selected trial composition, together with the properties measured for it, as an additional data point in an expanded training data set for retraining the neural network using the expanded training data set.

The input vector, which is formed on each iterative retraining on the basis of the properties determined by the facility, contains the empirically measured properties of a product produced according to the selected trial composition. The output vector contains an amount of predicted components of the selected trial composition, and so, using the loss function, the predicted components can be compared with the actual components of the trial composition. The input vector used for testing the loss function is preferably the same at each iteration, thereby allowing changes in the error value calculated by the loss function to be attributed to changes in the predictive model of the network and not to changes in the input vector. In some embodiments, the loss function is also employed for a plurality of test compositions having empirically known properties, in order to broaden the data pool when determining whether the loss function fulfils a particular criterion.

When the training of the network is concluded, the trained neural network is able to predict, generate and output a forecast composition on the basis of an input vector which specifies a plurality of desired properties and corresponding parameter value ranges. This information may be output to a user and/or to the facility, and may automatically cause the facility to produce a chemical product in accordance with the forecast composition and to test empirically whether it has the desired properties.

Very generally, the method described may be advantageous particularly for calculating a forecast composition in the context of the production of mechanically and/or thermally loaded component parts, since predicting a suitable composition is barely possible on the basis of the multitude of components and their interactions.

For example, it is possible in the case of electrically conductive thermoplastics to add various auxiliaries, e.g. conductive particles such as carbon black, carbon nanotubes (CNTs), graphene, etc., or salts, e.g. ionic liquids, complex salts, etc. Such compositions are then characterized via their conductivities or resistances, e.g. breakdown resistance, surface resistance. The thermoplastics, which fundamentally are insulators (materials of very high electrical resistance), are therefore given limited conductivity through addition of various classes of substance, and the at least two additions both act to increase the conductivity. Moreover, these additions of course also influence other properties, for instance the viscosity, or they diverge greatly in cost. The additions may further be synergistically active.

FIG. 2 shows a block diagram of a distributed system 200 for training a neural network 226 and for using the trained network to predict compositions, especially thermoplastic compositions for mechanically and/or thermally loaded component parts.

The system comprises a database 204 featuring known compositions 206 and also featuring trial compositions 208. As already described above, the database may at its most simple consist of a memory region storing one or more files, text files or comma-separated files, for example. In the case of the embodiment shown in FIG. 2, the database 200 is the database of a database management system (DBMS), for example of a relational DBMS such as MySQL, for example, or object-oriented DBMS such as JSON, for example. The use of a relational or object-oriented DBMS is especially advantageous with relatively large data sets, for the administration and rapid searching of the data sets, allowing searches to be specified more accurately and performed more quickly. For example, the known compositions 206 may be stored in a first database table and the trial compositions 208 in a further database table. It is also possible, however, for all known compositions and trial compositions to be stored in a single table and for the different types to be labelled accordingly in data sets by metadata/“flags”. The way in which the data is filed may be freely selected by the skilled person, provided that the type of storage permits a logical differentiation between the two types of composition.

The known compositions 206 may comprise, for example, an amount of data sets which comprises in each case a composition which has already been actually used at least once to produce a corresponding chemical product, and the physical, chemical, tactile, optical and/or other metrologically capturable properties of this product. For example, the known compositions 206 may be the entirety of those compositions which have already been produced before by a particular organization, or by a particular laboratory, or by a particular laboratory facility 244, and of which also at least some of the aforementioned metrologically capturable parameters (properties) have been captured empirically.

The known compositions 206 stored in the database 204 are therefore distinguished by the fact that not only their components (that is, the individual chemical constituents and their respective amounts and/or concentration details), but also at least some metrologically capturable properties of the chemical product produced according to this composition, are known. Every known composition of a chemical product is therefore represented in the database 204 as a data set which comprises the components of this product and also the said metrologically captured properties of this product.

The trial compositions 208 stored in the database 204, on the other hand, are compositions whose physical, chemical, optical and/or other metrologically capturable properties are unknown at least to the operator of the database and/or of the laboratory facility 244. This is indicated by question marks in FIG. 2. In the database 204, therefore, a trial composition is represented as a data set which does indeed characterize the components (that is, the individual chemical constituents and optionally their respective amounts and/or concentration figures as well) of a chemical product, but does not characterize the said metrologically capturable properties of this product. For example, the stated properties may not be present because the composition in question has never yet been used, or at least not by the laboratory or laboratory facility in question, to synthesize a corresponding chemical product.

In some embodiments, the trial compositions 208 may be compiled manually by a skilled person and stored in the database. For example, a chemist is able, based on their experience in the production of mechanically and/or thermally loaded component parts and their respective properties, to specify new trial compositions which the skilled person expects to have these particular desired materials properties. The trial compositions may be generated, for example, by the skilled person modifying a known composition by omitting or newly adding individual components. If the composition also comprises concentrations of the one or more components, and the production parameters (“compound”), an extended trial composition of this kind may also be formed by changing the concentrations of the components in known compositions.

In other embodiments, the trial compositions 208 are compiled automatically and stored in the database 204. Each of the known compositions 206, for example, may consist of 20 different chemical components. The trial compositions 208 are then generated automatically by replacing individual components in the composition with other substances.

If the compositions are “extended” compositions with concentration details, then trial compositions can also be formed by varying the amounts of individual components in the known compositions 206, i.e. for example increasing them by 10% and/or lowering them by 10%. If in each case only a single component is ever varied, by a 10% increase and also a 10% reduction in the amount of that component used, two variants are therefore formed per component. In the case of 20 components, then, this method gives rise to 40 trial compositions. The number of trial compositions generated automatically is preferably increased still further by a simultaneous 10% increase or decrease in the concentration of two or more components relative to their concentration in the known composition. Purely combinatorially, 2²⁰=1048576 extended trial compositions can be generated automatically in this way. The number of trial compositions may additionally be increased very greatly by using even more concentration variants, i.e., for example, −20%, −10%, +10%, +20%, for each of the 20 components, and/or by omitting or additionally using chemical components. The amount of the trial compositions may therefore be very high, especially in the case of automatic generation of the trial compositions.

In some embodiments, the trial compositions 208 include not only manually compiled trial compositions but also automatically generated trial compositions.

For example, automatic generation of the trial compositions may be advantageous since it allows coverage, in a rapid way, of a very large parameter space made up of components and optionally their concentrations as well, this parameter space typically covering the individual components and their concentrations broadly and in a coarse-meshed manner, when using a corresponding algorithm to generate the trial compositions.

The manually supplemented trial compositions may be trial compositions which to a skilled person on the basis of their empirical knowledge promise a particularly high learning effect for the neural network, or from whose synthesis the skilled person, for other reasons, expects advantageous insights.

In FIG. 2, the generally large number of trial compositions is indicated by the fact that the database 204 encompasses only 100 known compositions but 900 trial compositions. The actual numerical ratio between known compositions and trial compositions, however, is heavily dependent on the particular case—in other words, for example, on how many compositions have already been generated and have their chemical properties determined by a particular laboratory, whether the database has integrated known compositions and their properties from external sources, and/or whether the trial compositions have been compiled manually or automatically. It is entirely possible, consequently, for the database 204 to include several 1000 known compositions. The amount of the trial compositions is typically considerably greater than the amount of empirical synthesis trials which a laboratory can actually carry out physically with an eye to costs and profitability.

The distributed system 200 further comprises a computer system 224, which comprises a neural network 226 and an active learning module 222. The active learning module 222 has access to the database 204. The access comprises at least a read access, in order to be able to read out one or more selected trial compositions and their components from the database 204. According to some embodiments, the active learning module and/or a chemical facility 244 which produces a chemical product in accordance with the selected trial composition and subjects it to metrological analysis also has/have writing rights to the database 204, in order to store the properties captured metrologically for the selected trial composition in the database. For example, the storage of the metrologically captured properties of a selected and newly synthesized trial composition may lead to this trial composition becoming a known composition and correspondingly, in the database 204, being stored at a different location and/or provided with different metadata (“flags”). For example, the DBMS 202 may be installed on a database server, so that the access to the database 204 by the active learning module and/or the chemical facility 244 is via a network. The network may more particularly be the Intranet of an organization, or else the Internet.

Other system architectures are also possible. For example, the database 204 and/or the DBMS 202 may also be part of the computer system 224 or of the main control computer 246, and/or the neural network 226 and the active learning module 222 may be installed on different computer systems. Independently of the architecture actually chosen, there must be a possibility for exchange of the data 210, 212, 214, 218 as represented in FIG. 2, for example, such that all of the components forming part of the method are able to obtain the required input data from other components. The data exchange may be direct or indirect via further components such as gateways, for example. In some embodiments, for example, the chemical facility can store the properties captured metrologically for the selected trial composition directly into the database 204, or send them only to the computer system 224, which then stores the properties in the database 204 in such a way that the data set of the trial composition is supplemented by the properties so as to become a “known composition”.

According to another alternative system architecture, the computer system 224 and the main control computer 246 are the same computer system.

According to embodiments of the invention, the computer system 224, or the components 226, 222 installed thereon, is/are designed first to read out the known compositions 206 from the database and to use the known compositions 206 as the training data set 210 for the initial training of the neural network 226. In the course of the training of the neural network, a predictive model of the neural network is generated, and this model, based on the training data set, models the relationships between the components used to synthesize a chemical product (i.e. between a composition) and the metrologically captured properties of the produced product. With the aid of this predictive model obtained in the course of the training, the trained neural network is able, on the basis of desired properties input in the form of an input vector, to predict the composition that specifies a product prospectively having the desired properties. An example of a possible desired property is that the conductivity of the chemical product lies within a certain parameter value range.

Because of the limited number of the known compositions 206 and their properties, the trained neural network 226 obtained after the initial training phase is in some cases not yet able to predict with sufficient reliability the components of a composition based on a list of desired properties. The data pool used is often too small for this to be the case.

Expanding the training data set by producing all of the trial compositions in the facility 244 and thereafter metrologically determining their properties is usually too expensive and/or too complicated. According to embodiments of the invention, the use of the active learning module makes it possible in a targeted way for a few trial compositions to be selected, and a corresponding chemical product synthesized and analyzed in the chemical facility 244 only for these compositions, so as to expand the training data set, with a very small number of syntheses (and hence with maximum efficiency), in such a way as to achieve a significant improvement in the predictive power of the predictive model of the neural network 226 by means of renewed training using the expanded training data set. After the renewed training of the neural network using the expanded data set, the predictive power of the neural network is again tested using the loss function 228. If in this test a mandated criterion is not fulfilled, and if, for example, the loss value thus exceeds a mandated maximum value, this means that the quality of the neural network or its predictive model is still not yet high enough, and there should be further training using an even more greatly expanded training data set. In that case, the active learning module carries out a further selection step in relation to a trial composition which has so far not been selected and used for synthesis of the corresponding medium. As described, a chemical product is synthesized on the basis of the trial composition selected, and the properties of this product are captured metrologically, so that the selected trial composition, together with the measured properties, can be added as a new training data set to the existing training data, in order to retrain the neural network 226 on the basis of an even more greatly expanded training data set. In a plurality of iterations, therefore, through automatic and targeted selection of trial compositions which promise a particularly high level of improvement in the predictive model of the neural network, and also by automatically performed steps of synthesis and analysis on the basis of the particular trial composition selected, it is possible to achieve rapid improvement in the predictive power of the neural network in an efficient way. If it is found after an iteration that the loss function meets the criterion, there is no longer any need for the training data set to be expanded, because the predictive power of the trained neural network can be considered to be sufficient.

Identifying the trial composition to be selected may be accomplished, for example, as shown in FIG. 2. The active learning module 222 is configured to read out the identified trial composition from the database 204 (e.g. by means of a SELECT command 214 to a relational database) and transmit it to the chemical facility 244. The computer system 224 therefore includes optionally an interface for communication with the facility 244 and/or is part of the facility. The facility is configured to synthesize a chemical product on the basis of the selected trial composition 212 and to measure one or more properties of the produced product. For example, the facility may comprise a plurality of synthesis devices 254, 256 or synthesis modules and a plurality of analysis devices 252 (or analysis modules) each of which carries out one or more steps in the synthesis or analysis of chemical products or their intermediates. The facility further possesses optionally one or more transport units 258, for example conveyor belts or robot arms, which transport the components, intermediates and consumables back and forth between the various synthesis and/or analysis units. The facility 244 encompasses a main control computer 246 with control software 248, or is operatively coupled to such a computer 246 via a network. The control software 248 is configured to coordinate the synthesis, analysis and/or transport steps, carried out by the synthesis and analysis units and optionally by the transport unit, in such a way that a chemical product is produced in accordance with the information in the selected composition 212, and its properties are captured metrologically. The control software preferably stores the captured properties 218 of the newly produced selected composition (directly or through the mediation of the computer 224) in the database 204 in such a way that the properties as stored are linked with the selected trial composition. In this case, then, the “incomplete” data set of the selected trial composition is supplemented to include the properties captured metrologically in the facility 244, and thereby transformed into a “known composition”.

Moreover, the properties 218 of the selected trial composition are transmitted to the computer system 224, and so a combination of the composition 212 selected by the active learning module and the properties 218 of that composition produces a new, complete data set, extending the entirety of the training data. On the basis of the extended training data set, the neural network is newly trained, and the effect of the extension of the training data on the quality of the prediction of the neural network is tested by means of the loss function 228. If the value of the loss function meets a pre-defined criterion, that is, for example, if it only has a loss value which is below a maximum value, the training can be ended. Otherwise, the result of the criterion testing is transmitted to the active learning module, which is caused to select a further trial composition.

According to some embodiments, the facility 244 or else a plurality of facilities for the synthesis and analysis of chemical products is or are also part of the distributed system 200.

The facility 244 may be a high-throughput experimentation facility, as for example a high-throughput facility for production and analysis of thermoplastic polymers and component parts. For example, the facility may be a system for the automatic testing and automatic production of chemical products, of the kind which is described in WO 2017/072351 A2.

The skilled person, therefore, using the system shown in FIG. 2, is able to avoid the need for a multiplicity of iteration stages and component compositions to be produced and analyzed, in an untargeted and complicated way, in order to obtain a sufficiently large training data set. Because of the massive complexity of the relationships between components, their respective combinations and concentrations, and also the complexity of the relationships between the various metrologically capturable properties of chemical products, their components and their concentrations, it is generally almost impossible for a skilled human to mentally grasp all of these relationships in their entirety and, in a targeted way, to carry out manual specification of highly promising compositions. Faced with the immense size of the combinatorial possibility sphere of components and concentrations, a skilled human can also only ever actually carry out empirical testing on a comparatively small and more or less randomly chosen segment of this possibility sphere. To date, therefore, it has been unavoidable to employ a lot of time and materials on the synthesis and analysis of compositions which ultimately have unwanted product properties and/or whose use as part of a training data set did not provide any notable improvement in the quality of a prediction model of a neural network. Through the use of an active learning module for the targeted selection of a few trial compositions, the operation of providing a suitable training data set for developing an accurate neural network can be accelerated considerably.

In a further advantageous aspect, according to embodiments of the invention, the system 200 can be used to provide a training data set which, through targeted selection of trial compositions, provides optimum supplementation of an existing, historical set of known compositions. The known compositions 206 may be compositions and their properties which have been produced and analyzed in the course of the operation of a laboratory or a laboratory facility, with the corresponding data having been stored. Possibly, therefore, the known compositions are not uniformly distributed over the combinatorially possible sphere of components and optionally concentrations as well, but instead result randomly from the history of the operation of the laboratory or facility. The active learning module may be used and configured to supplement the predictive model of the neural network, formed initially on the basis of the initial training using the known compositions 206, by a few further experimental syntheses and analyses, in such a way that, for example, the components and concentrations covered only inadequately by the known compositions 206 are now covered by means of the targetedly selected trial compositions.

When the iterative training of the neural network has ended, the trained neural network can be used to predict compositions (“forecast compositions”) which have certain metrologically capturable properties, and which are therefore located within a defined and desired value range in terms, for example, of a chemical or physical or other parameter. For this purpose, the desired properties are presented as an input vector and are input into the trained neural network. The neural network then ascertains the nature of the components (and optionally their amount as well), and optionally also the production conditions, that can be used to generate a chemical product having the desired properties. The forecast composition can be transmitted by the neural network automatically to the facility 244, together with a control command to generate a chemical product in accordance with the forecast composition. The control command may optionally also cause the facility to carry out automatic measurement of properties of this product and to store the forecast composition in the database together with the properties obtained for said composition, and thereby to expand the amount of known compositions.

The various components of the system, insofar as this has not been expressly noted, need not be locally tied. Computer systems and their components, as is known, may be distributed worldwide, but in the context of the invention are each considered as a unit.

FIG. 3 shows a 2D detail of a multi-dimensional data space 300, from which the active learning module carries out targeted selection of data points 308 for expanding the training data set. In the course of the training, the neural network, on the basis of an input vector which specifies the properties of a composition, learns to calculate an output vector which represents a composition, i.e. a specification of the components of a chemical product. According to embodiments of the invention, the properties specified in the input vector include, in particular, the following properties and also combinations of two or more of these properties: amino end-group content, carboxyl end-group content, breakdown resistance, melt-volume flow rate (MVR), glass transition temperature, combustibility (UL94), notched impact toughness, breaking stress, elongation at break, yield stress, elasticity modulus, heat resistance. The cost reduction during production may be captured, for example, by a chemical facility automatically during synthesis of a composition, and may be based, for example, on a mandated reference value. It is, however, also possible for a human to capture the costs manually. In general, the only properties that can be specified in the input vector are those which were also part of the training data set used to train the neural network.

After the neural network has undergone initial training, it has already “learnt” certain relationships between components of the compositions and some properties, based on the known compositions. These learnt relationships are illustrated here by the parting line 316, which divides the data space 300, in respect of the property of “conductivity”, into a data space 320 having electrically unacceptable product properties 320, on the left below the parting line 316, and a data space 318 having electrically acceptable product properties, on the right above the parting line. FIG. 3 is able only to represent a component aspect of the data space 300, confined to two dimensions, that is to say correspondingly two components (“concentration of conducting salt” and “concentration of CNT”), one property and one component, or two properties. The data space 300 is per se multi-dimensional, and may, for example, with 20 components, have 190 corresponding two-dimensional relationships (binomial function: n over 2), with each of these spaces formed by said 190 relationships containing independent parting lines or multi-dimensional parting planes (“hyperplanes”) in respect of the particular property under consideration.

The data points, shown as circles in FIG. 3, represent in each case one of the trial compositions 208. One of the trial data points may be selected according to the “minimum marginal hyperplane” approach. For example, the active learning module may take the form of a support vector machine or another algorithm which is capable of dividing a data space, generated by the trial compositions, into sub-spaces in relation to one or more properties (or components), on the basis of the predictive model already learnt by the neural network 226. The model already learnt by the neural network is therefore represented here by the parting line or parting plane 316. The basis for the “minimum marginal hyperplane” method is that the data points with the smallest distance from the parting line 316 are those for which the predictive model already learnt, and indeed represented by this parting line 316, is the least sure, and therefore that the trial composition belonging to this data point ought to be selected, produced and analyzed in order to provide empirical determination of the actual properties—in this case, for example, the electrical conductivity. In the example represented here, the active learning module, taking account solely of the property of “electrical conductivity”, would therefore select the trial composition represented by the data point 308. The chemical facility 244 would be initiated to synthesize and analyze this composition (represented by data point 308), in order to expand the training data by the components of this trial composition and its empirically measured properties and to improve the neural network by training it using the expanded training data set. It may, for example, be the case that empirical measurement of the composition represented by the point 308 reveals that its electrical conductivity lies within the unacceptable region 320. Accordingly, retraining using the expanded training data set would result in the predictive model of the neural network, visualized graphically here by the parting line 316, adapting itself in such a way that for a composition like that represented by point 308, the prediction in future is that its electrical conductivity lies within the region 320. As a result of the renewed training using the expanded training data set, therefore, the parting line/parting plane 316 would be modified such that the line or plane receives a “swelling” to the top right, so that the improved neural network would now have recognized and predicted that the composition represented by point 308 lies within the electrically unacceptable region 320. In practice, in the selection of the data point and of the corresponding trial composition, the distance of the corresponding data points from the parting lines of two or more properties is preferably taken into account—for example, by selection of the data point having the minimum average distance from all parting lines/parting planes of the data space 300.

FIG. 4 shows the architecture 400 of a trained neural network which is configured and trained to receive an input vector 402 as an input and from it to calculate and output an output vector 406. The input vector 402 specifies the desired properties or the corresponding parameter value ranges of a composition whose components (and optionally the concentrations or amounts of the respective components as well) are to be predicted (forecast) by the neural network. The output vector 406 specifies the components of a forecast composition and optionally the amounts or concentrations of these components in the forecast composition as well, a forecast composition being a composition predicted by the neural network to have properties lying within the parameter value ranges mandated in the input vector. The network comprises a plurality of layers 404 of neurons which are linked, by means of weighted mathematical functions, with the neurons of other layers in such a way that the network, on the basis of the desired properties specified in the input vector, is able to calculate, and thus predict, the components of the corresponding composition and is able to output these components, and hence the forecast composition itself, in the form of an output vector 406.

Prior to the training, the neurons of the neural network are first of all initialized with mandated or random activations (weights). During the training, the network receives an input vector which represents empirically measured properties of a known composition, calculates the output vector with predicted components (and optionally amounts of components) of this composition, and is penalized by the loss function for deviations of the predicted components from the components actually used. The prediction error ascertained is distributed back, via a process called back-propagation, to the respective neurons which gave rise to the error, with the effect that the activations (weights) of certain neurons change in such a way that the prediction error (and hence the value of the loss function) is reduced. For this purpose, viewed mathematically, the slope of the loss function can be determined, and so the activations of the neurons can be modified in a directed way so as to minimize the value output from the loss function. As soon as the prediction error or loss function value is below a pre-defined threshold, the trained neural network is regarded as being sufficiently precise, and so there is no need for further training.

For example, the task may be that of generating a new, unknown composition which has a defined electrical conductivity in the value range EVR, a defined color in the value range CVR and an abrasion resistance in the value range AVR. Before this composition is produced for real in the laboratory, the neural network is to be used initially to ascertain, automatically, the components of a composition which specifies the components of a chemical product whose electrical conductivity, color and abrasion resistance lie within the desired value ranges EVR, CVR and AVR. If no forecast composition is found with properties which lie within the desired value ranges, it is possible to abandon the synthesis straight away and save costs. It may make sense here to alter the mandates in relation to the properties.

The components of this new composition and optionally their respective desired concentration as well are output as the output vector 406 of the neural network to a user for manual evaluation and/or to a chemical facility. The output vector may contain, for example, 20 components of a forecast composition which has been forecast by the neural network to have, or for the chemical product produced in accordance with the forecast composition to have, the desired properties. The input properties are properties also already considered during the training of the neural network. In other embodiments, depending on the nature of the composition and/or on the properties considered to be relevant, the vectors 402, 406 may also comprise a higher or lower number of elements.

The method of the invention for producing a thermoplastic composition for mechanically and/or thermally loaded component parts also encompasses the version whereby the formulation and production instructions have been drawn up as described above and the industrial production takes place on a facility different from the facility described above. The term “industrial” refers to versions wherein, as polymer, 10 kg, preferably 50 kg, more preferably 100 kg, especially preferably 500 kg, or more are processed.

The invention has as a further subject thermoplastic compositions for mechanically and/or thermally loaded component parts, produced by the methods of the invention, explicitly by the method whereby the composition takes place according to the industrial production.

### LIST OF REFERENCE NUMERALS

102-118 Steps
200 Distributed system
202 DBMS
204 Database
206 Known compositions with properties
208 Trial compositions (properties unknown)
210 Training data set originally used
212 Selected trial composition
214 Selection command for a trial composition
218 Empirically determined properties of the selected trial composition
222 Active learning module
224 Computer system
226 Neural network
228 Loss function
244 Chemical facility
246 Main control computer
248 Control software
252 Analysis device
254 Synthesis device
256 Synthesis device
258 Transport unit
300 2D detail of a multi-parameter data space of the trial compositions
302-312 Data points (each representing a trial composition)
316 Parting line of the predictive model of the trained neural network
318 Rheologically acceptable region
320 Rheologically unacceptable region
400 Architecture of the neural network
402 Input vector
404 Layers of the neural network
406 Output vector

1. A method for generating a thermoplastic composition for mechanically and/or thermally loaded component parts,
wherein the components of the composition have one or more polymers selected from the group consisting of polyamides, polyesters, polyolefins, polycarbonates and polyaryletherketones,
where the composition is generated by a computer system (224), where the computer system has access to a database (204), where known compositions (206) are stored with their components and properties in the database, and where the computer system is connected to a facility (244) for producing and testing compositions for mechanically and/or thermally loaded component parts, where the computer system comprises a neural network (226) and an active learning module (222),
the method comprising the following steps:
a. using (102) of known compositions (206) stored in the database for training the neural network (226), where a loss function (228) is minimized for the training,
b. testing (104) to determine whether the value of the loss function meets a specified criterion,
where selectively, in the event that the criterion is not met, the following steps are carried out:
i. selecting (106) of a trial composition (212) from a quantity of specified trial compositions (208) by the active learning module (222),
ii. driving (108) of the facility (244) by the computer system for producing and testing the selected trial composition,
iii. training (110) of the neural network using the selected trial composition (212) and the properties (218) thereof captured by the facility,
iv. repeating implementation (112) of step b,
c. generation (116) of a forecast composition (406) for mechanically and/or thermally loaded component parts by input of an input vector (402) into the neural network (226),
d. output (118) of the forecast composition (406) by the neural network (226).

2. The method according to claim 1, wherein the quantity of the trial compositions (208) is generated automatically by a trial planning program.

3. The method according to claim 1, wherein in addition to the polymers further components are selected from the group consisting of processing assistants; nucleating agents and demoulding aids; stabilizers; colorants; conductivity assistants (electrical); thermal conductivity additives; reinforcing agents; impact modifiers; fillers; flame retardants; and plasticizers.

4. The method according to claim 1, wherein the properties are selected from the group consisting of notched impact toughness, bursting periphery tension, BET surface area, amino end-group content, carboxyl end-group content, density, dielectric constant, conventional bowing, failure mode, yield stress, breakdown resistance, maximum penetration force, number of fractures—ball drop and hammer drop tests, color number L*ab, pull-out force (fittings), corrected intrinsic viscosity (CIV), permeation (time), melt-volume flow rate (MVR), oxygen index, surface resistance, melt viscosity, end-group total, melting temperature, enthalpy of fusion, glass transition temperature, crystallinity, dimensional change, tapped density, transmittance, combustibility (UL94), Vicat softening temperature, heat distortion resistance temperature and water content, elongation at break, yield stress, elasticity modulus, heat resistance.

5. The method according to claim 1, wherein the forecast composition is output on a user interface of the computer system.

6. The method according to claim 1, wherein the facility (244) comprises at least two workstations (252, 254, 256), where the at least two workstations are connected to one another via a transport system (258) on which transport vehicles are able to run for transporting the components of the composition and/or the composition produced between the workstations, where the method further comprises:
input of a composition to a processor which controls the facility (244), where the composition input into the processor is the selected trial composition (212) or the forecast composition, where the processor drives the facility to produce the input composition, where in the at least two workstations the input composition is produced and the properties of the input composition are measured, after which the measured properties are output on a user interface of the computer system and/or the measured properties are stored in the database (204).

7. The method according to claim 1, wherein the computer system (224) communicates via a communications interface with the database (204) and/or with the facility (244) for producing and testing compositions for mechanically and/or thermally loaded component parts, where the communications interfaces are selected from SCSI, USB, FireWire, Bluetooth, Ethernet, WLAN, LAN or are realized by another network interface.

8. The computer system (224) for generating a composition for mechanically and/or thermally loaded component parts, comprising a database and a user interface, where the computer system is configured for implementing a method according to claim 1.

9. The computer program, digital storage medium or computer program product with instructions which can be executed by a processor in order to implement a method according to claim 1.

10. The system (200) comprising
a facility (244) for producing and testing mechanically and/or thermally loaded component parts, where the facility comprises at least two workstations, where the at least two workstations are connected to one another optionally via a transport system on which transport vehicles are able to run for transporting the components of the composition and/or the composition produced between the workstations, and
a computer system (224) according to claim 9.

11. The method for producing a thermoplastic composition for mechanically and/or thermally loaded component parts, wherein the formulation and production instructions have been drawn up as in claim 1 and the industrial production takes place on a different facility.

12. The thermoplastic composition for mechanically and/or thermally loaded component parts, produced by the method according to claim 1.

13. The method according to claim 2, wherein in addition to the polymers further components are selected from the group consisting of processing assistants; nucleating agents and demoulding aids; stabilizers; colorants; conductivity assistants (electrical); thermal conductivity additives; reinforcing agents; impact modifiers; fillers; flame retardants; and plasticizers.

14. The method according to claim 2, wherein the properties are selected from the group consisting of notched impact toughness, bursting periphery tension, BET surface area, amino end-group content, carboxyl end-group content, density, dielectric constant, conventional bowing, failure mode, yield stress, breakdown resistance, maximum penetration force, number of fractures—ball drop and hammer drop tests, color number L*ab, pull-out force (fittings), corrected intrinsic viscosity (CIV), permeation (time), melt-volume flow rate (MVR), oxygen index, surface resistance, melt viscosity, end-group total, melting temperature, enthalpy of fusion, glass transition temperature, crystallinity, dimensional change, tapped density, transmittance, combustibility (UL94), Vicat softening temperature, heat distortion resistance temperature and water content, elongation at break, yield stress, elasticity modulus, heat resistance.

15. The method according to claim 2, wherein the forecast composition is output on a user interface of the computer system.

16. The method according to claim 2, wherein the facility (244) comprises at least two workstations (252, 254, 256), where the at least two workstations are connected to one another via a transport system (258) on which transport vehicles are able to run for transporting the components of the composition and/or the composition produced between the workstations, where the method further comprises:
input of a composition to a processor which controls the facility (244), where the composition input into the processor is the selected trial composition (212) or the forecast composition, where the processor drives the facility to produce the input composition, where in the at least two workstations the input composition is produced and the properties of the input composition are measured, after which the measured properties are output on a user interface of the computer system and/or the measured properties are stored in the database (204).

17. The method according to claim 2, wherein the computer system (224) communicates via a communications interface with the database (204) and/or with the facility (244) for producing and testing compositions for mechanically and/or thermally loaded component parts, where the communications interfaces are selected from SCSI, USB, FireWire, Bluetooth, Ethernet, WLAN, LAN or are realized by another network interface.

18. The method for producing a thermoplastic composition for mechanically and/or thermally loaded component parts, wherein the formulation and production instructions have been drawn up as in claim 2 and the industrial production takes place on a different facility.

19. The thermoplastic composition for mechanically and/or thermally loaded component parts, produced by the method according to claim 2.

20. The thermoplastic composition for mechanically and/or thermally loaded component parts, produced by the method according to claim 11.
